(19) United States
(12) Patent Application Publication
(10) Pub. No.: US 2008/0119770 A1
(43) Pub. Date: May 22, 2008

(54) SLING FOR SUPPORTING AN ARM

(76) Inventor: DREW V. MILLER, Atlanta, GA (US)

Correspondence Address: CHRISTOS S. KYRIAKOU, 2055 AZALEA DRIVE, ROSWELL, GA, 30075, US

(21) Appl. No.: 11/939,539

(22) Filed: Nov. 13, 2007

Related U.S. Application Data
(60) Provisional application No. 60/860,402, filed on Nov. 21, 2006.

(51) Int. Cl. A61F 5/37

(52) U.S. Cl. 602/4

(57) ABSTRACT

A sling for supporting a portion of an arm of an individual that includes: a means for receiving and supporting a portion of an arm of an individual, the means for receiving and supporting a portion of an arm of an individual having a posterior portion and an anterior portion; a means for securing the sling to an individual, the means for securing the sling to an individual designed and adapted to encircle a shoulder, the means for securing the sling to an individual having a front-facing side and a back-facing side; a first means for attaching the means for receiving and supporting a portion of an arm of an individual to the means for securing the sling to an individual; and a second means for attaching the means for receiving and supporting a portion of an arm of an individual to the means for securing the sling to an individual is provided.

### CROSS-REFERENCE TO RELATED APPLICATIONS

This application claims the benefit of priority of U.S. provisional application Ser. No. 60/860,402, entitled “Sling for Supporting an Arm”, filed on Nov. 21, 2006 by Drew V. Miller, the content of which is relied upon and incorporated herein by reference in its entirety.

### BACKGROUND OF THE INVENTION

1. Field of the Invention

The present invention relates to orthopedic devices. More particularly, the present invention relates to slings for supporting at least a portion of an arm of an individual.

2. Description of the Related Art

Physicians frequently treat arm and shoulder injuries with a sling to at least partially support and/or immobilize the arm of a wearer of the sling. For example, a physician will request a patient to wear a sling after shoulder surgery or after diagnosing a shoulder or an arm injury so that the shoulder and/or the arm will remain relatively motionless while the patient heals. Typically, a sling consists of a pouch for receiving an elbow and a forearm and a strap having one end attached to one end of the pouch, a middle section that wraps around the neck and shoulder of the wearer with the other end of the strap attached to the other end of the pouch. A disposable example of such a sling is illustrated and described in U.S. Pat. No. 7,018,349. Unfortunately, such simple slings are uncomfortable and tend to creep or rotate backward about the spinal or vertical axis of a wearer. A more complicated attempt to provide an improved shoulder sling is described and illustrated in U.S. Patent Application No. 2005/0010147.

Current slings are not very comfortable because the strap rests on the neck of the wearer, tends to ride up the neck of the wearer and cause the wearer to support the weight of the arm on or near the neck. Thus, there is a need for an improved sling that is simple and easy to use and that is more comfortable and provides greater stability. The present invention provides a sling that meets one or more of the above-mentioned shortcomings.

### BRIEF SUMMARY OF THE INVENTION

The present invention provides a sling for supporting a forearm that includes: a pouch for receiving and supporting a forearm, the pouch having a posterior portion and an anterior portion; an encircling strap designed and adapted to encircle a shoulder, the encircling strap having a front-facing side and a back-facing side; a first chest strap having a first end connected to the front-facing side of the encircling strap and a second end connected to the anterior portion of the pouch; and a back strap having a first end connected to the back-facing side of the encircling strap and a second end connected to the posterior portion of the pouch. Desirably, the sling impedes creeping or movement of the pouch about the spine or vertical axis of the wearer. In certain desirable embodiments, the encircling strap is designed and adapted to encircle the shoulder from the clavicle across an upper portion of the chest of a wearer below an armpit of the wearer and across an upper portion of the back of a wearer back to the clavicle and rests on a region between the neck and the joint of an arm and a shoulder of a wearer of the sling to keep the encircling strap away from the neck of the wearer. For example, the encircling strap is desirably designed and adapted to fit no closer than about 2 centimeters from the neck of the wearer, more desirably no closer than about 3 centimeters from the neck of the wearer, still more desirably no closer than about 4 centimeters from the neck of the wearer, and still more desirably no closer than about 5 centimeters from the neck of the wearer. In certain instances, the encircling strap is designed and adapted to fit no closer than about 6 centimeters from the neck of the wearer and even no closer than about 7 centimeters from the neck of the wearer.

The sling may further include an optional shoulder pad designed and adapted to rest on the wearer's shoulder at a point of contact of the encircling portion and the clavicle of the wearer. The encircling strap may also include an optional buckle, a hook portion and a loop portion or another means for adjusting the circumference of the encircling strap. The first chest strap and or the back strap may also include an optional buckle, a hook portion and a loop portion or another means for adjusting the supporting length of the first chest strap in certain embodiments, the sling also includes a second chest strap having a first end connected to the front-facing side of the encircling strap, a second end connected to the anterior portion of the pouch and a middle portion supported by the first chest strap. The encircling strap may consist essentially of a single strap, may include two dog leg bends or may also include an optional buckle, a hook portion and a loop portion or another means for adjusting the circumference of the encircling portion.

### BRIEF DESCRIPTION OF TUE DRAWINGS

A full and enabling disclosure of the present invention, including the best mode thereof is set forth in the detailed description which makes reference to the appended figures in which:

FIG. 1 is a front perspective view of one embodiment of a sling of the present invention; and

FIG. 2 is a front perspective of the sling of FIG. 1 properly applied to a wearer.

### DETAILED DESCRIPTION OF THE INVENTION

The drawings and detailed description provide a full and detailed written description of the invention, and of the manner and process of making and using it, so as to enable one skilled in the pertinent art to make and use it, as well as the best mode of carrying out the invention. The detailed description uses numerical and letter designations to refer to features in the drawings. Like or similar designations in the drawings and description have been used to refer to like or similar parts of the invention. The examples set forth in the drawings and detailed description are provided by way of explanation of the invention and are not meant as limitations of the invention. The present invention thus includes any modifications and variations of the following examples as come within the scope of the appended claims and their equivalents.

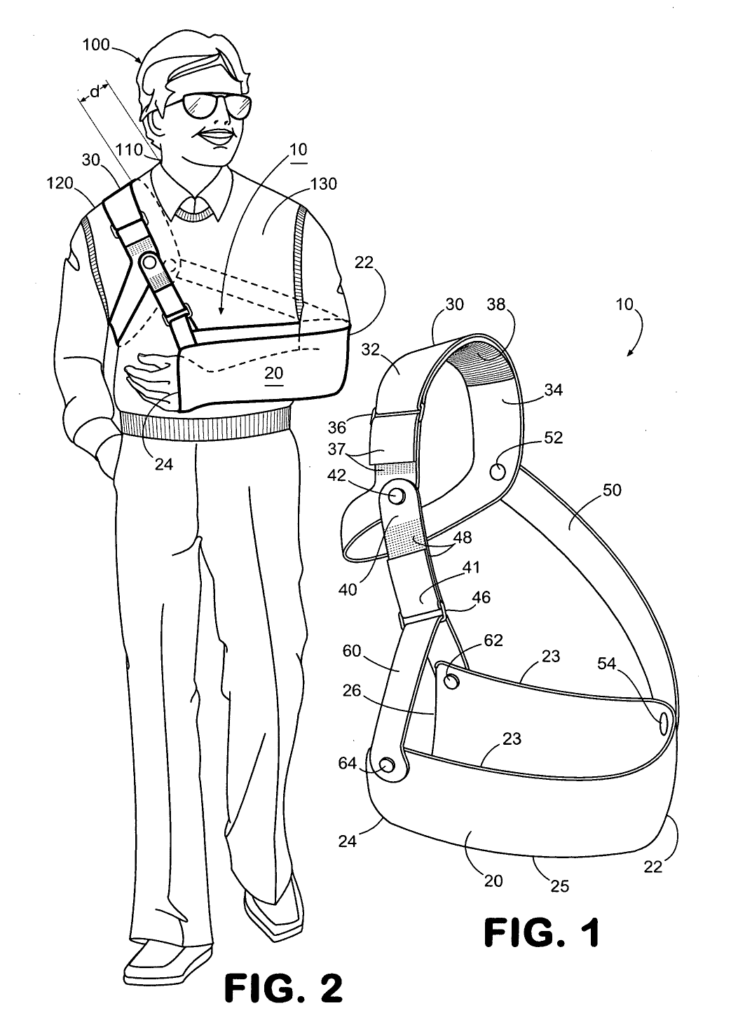

In the embodiments illustrated and described herein, a sling 10 is provided for receiving and supporting at least a portion of an arm of a wearer, for example as illustrated in FIG. 2. A front perspective view of one preferred embodiment of a sling of the present invention is provided in FIG. 1. The sling 10 includes a pouch 20 or other means for receiving and supporting a forearm or a portion of the arm of an individual. The pouch 20 may be of any shape that is capable of supporting a forearm or a portion of a forearm of a human. Pouches for receiving and supporting a forearm of a human are known and generally include a posterior portion 22 into which the elbow of a wearer is placed and an anterior portion 24 for receiving and supporting the forearm and even the wrist of a wearer. Typically, the pouch is shaped like an envelope and is open at one end, the anterior edge, so that a hand may extend out of the pouch. The pouch 20 is usually open along the top edge 23 so that an arm may be easily inserted and removed from the pouch 20. Generally, the pouch should be closed along the bottom edge 25 and the anterior edge 22 so that the pouch can support a forearm or enough of a forearm to at least partially immobilize an arm and relieve stress on the shoulder. The pouch can be made from one piece of material or from multiple pieces of material and generally can be made of any material that is used to make such pouches. Suggested materials include, but are not limited to, woven fabrics, nonwoven fabrics, and films and laminates of such materials. Desirably, the pouch should be constructed of a material or materials that provide enough strength or rigidity to adequately support an arm yet that are flexible enough and breathable enough to provide comfort to the wearer. The selection of such materials can be varied by those of skill in the art to achieve desired properties.

The pouch 20 is supported by an encircling strap 30 or other means for securing the sling 10 to the torso 130 of an individual 100. Preferably encircling strap 30 or means for securing the sling to the torso of an individual is designed and adapted to encircle a shoulder, for example as illustrated in accompanying FIG. 1. Preferably, encircling strap 30 or means for securing the sling is designed and adapted to rest on a clavicle somewhere between the neck 110 of the wearer and the joint of an arm and a shoulder of the wearer. More preferably, encircling strap 30 or means for securing the sling is designed and adapted to rest on a clavicle at about the midpoint between the neck 110 of the wearer 100 and the joint 120 of the arm and the shoulder of the wearer. In the illustrated embodiment, encircling strap 30 or other means for securing the sling to the torso of an individual encircles the shoulder from the clavicle across an upper portion of the chest of a wearer below an armpit of the wearer and across an upper portion of the back of a wearer back to the clavicle, for example as illustrated in FIG. 2. Desirably, encircling strap 30 is designed and adapted so that the inside edge of the encircling strap 30 fits no closer than about 1 centimeter from the neck 110 of the wearer 100. More desirably, encircling strap 30 is designed and adapted so that the inside edge of encircling strap 30 fits no closer than about 2 centimeters from the neck 110 of the wearer 100, still more desirably no closer than about 3 centimeters from the neck 110 of the wearer 100, still more desirably no closer than about 4 centimeters from the neck 110 of the wearer 100 and possibly no closer than about 5, about 6 centimeters or about 7 centimeters from the neck 110 of the wearer 100′ This distance “d” from inside edge of the encircling strap to the neck of the wearer 100 may vary depending on the size of the individual but should preferably keep the strap away from the neck of the wearer 100.

The encircling strap 30 may be circular, oval, ovoid, elliptical or may be of a more complex shape such as an oval or circle with doglegs or bends, for example as illustrated in FIGS. 1 and 2, in order to make the encircling strap 30 more comfortable and ergonomic and/or to improve fit. In certain embodiments, the encircling strap 30 is either left handed or right handed and in other embodiments the strap 30 is symmetrical and can be adjusted for use on either the right or left shoulder. The encircling strap 30 may also include one or more optional pads to provide additional cushioning and comfort to the wearer of the sling 20. For example, an optional pad 38 may be provided on encircling strap 30 at the point of contact of the encircling strap 30 and the clavicle. The encircling strap 30 may also include one or more buckles 36 or other means for adjusting the circumference and/or fit of encircling strap 30. Examples of means for adjusting the circumference and/or fit of the encircling strap 30 include, but are not limited to, hook and loop fasteners 37 such as VELCRO, buttons, snaps and so forth.

Encircling strap 30 has a front-facing portion 32 and a back-facing portion 34 to which are attached straps or other means for attaching and supporting pouch 20. In the embodiment illustrated in FIG. 1, pouch 20 is attached or otherwise connected to back-facing portion 34 of encircling strap 30 with a back strap 50 having a first end connected to the back-facing side 34 of the encircling strap 30 at rivet 52 and a second end attached to or otherwise connected to the posterior portion 22 of the pouch 20 at rivet 54. Pouch 20 is supported at the anterior portion 24 by first chest strap 40 and an optional second chest strap 60 as illustrated in FIG. 1.

In the embodiment illustrated in FIG. 1, first chest strap 40 has a first end connected to the front-facing portion 32 of the encircling strap 30 at rivet 42 and a second end 41 that connects to the anterior portion 24 of the pouch 20 via buckle 46 and second chest strap 60. Specifically, in the illustrated embodiment first chest strap 40 has a second end having hook and loop fastener 48 wrapped about and attached to a buckle 46 that and connects to and supports second chest strap 60. Optional second chest strap 60 slides through buckle 46 and connects to pouch 20 at each end of strap 60. More specifically, second chest strap 60 is attached to or otherwise connected to the anterior portion 24 of pouch 20 at rivet 62 provided on the back side 26 of the anterior portion 24 and rivet 64 provided at the front side of anterior portion 24. The length of first chest strap 40 and/or optional second chest strap 60 may be adjusted to support pouch 20 in a generally horizontal position or another desired orientation. Alternatively, straps 40 and 60 and buckle 46 can be one piece. Straps 30, 40, 50 and 60 can be made of any material that can be used to make straps including, but not limited to, nylon webbing and can be connected to pouch 20 and to each other using rivets as shown or with buttons, sewn connections or any other means for connecting a fabric straps to other fabric straps and/or other fabric materials.

Desirably, all or some of connections 42, 52, 54, 62 and 64 are pivotable so that the fit of the sling and/or the position of the adjustable pouch 20 are more adjustable. In the embodiment illustrated in FIGS. 1 and 2 the encircling strap 30 consists essentially of one strap with two bends, specifically dog leg bends of about 30 degrees to about 60 degrees, to improve fit. Encircling strap 30 also includes a hook and loop attachment 37 to adjust the effective length and, thus, the circumference and the fit of the encircling strap 30. Encircling strap 30 may be of other shapes, for example encircling strap 30 can be circular or ovoid. First chest strap 40, back strap 50, encircling strap 30 and even optional second chest strap 60 may include one or more buckles, hook and loop portions or other means for adjusting the supporting length of the respective strap and/or fit of the sling 10 as well as the relative orientation of the pouch 20. It should also be noted that the encircling strap 30 or means for securing the sling to an individual may be made from one strap, more than one strap or made from a rigid plastic or other non-fabric material and combinations thereof and may include optional padding.

Slings of the present invention may be designed and shaped so that a sling is reversible and can be used to treat either a wearer's right shoulder or a wearer's left shoulder. However, in certain desirable embodiments, slings of the present invention are designed and shaped in a left-handed configuration that is designed and shaped to better and more comfortably support a left arm or in a right-handed configuration that is designed and shaped to better and more comfortably support a right arm.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

We claim:

1. A sling for supporting a forearm, the sling comprising:
a pouch for receiving and supporting a forearm, the pouch having a posterior portion and an anterior portion;
an encircling strap designed and adapted to encircle a shoulder, the encircling strap having a front-facing side and a back-facing side;
a first chest strap having a first end connected to the front-facing side of the encircling strap and a second end connected to the anterior portion of the pouch; and
a back strap having a first end connected to the back-facing side of the encircling strap and a second end connected to the posterior portion of the pouch.

2. The sling of claim 1 wherein the sling impedes creeping or movement of the pouch about the vertical axis of the wearer.

3. The sling of claim 1 wherein the encircling strap is designed and adapted to rest on a region between the neck and the joint of an arm and a shoulder of a wearer of the sling to keep the encircling strap away from the neck of the wearer wherein the encircling strap encircles the shoulder from the clavicle across an upper portion of the chest of a wearer below an armpit of the wearer and across an upper portion of the back of a wearer back to the clavicle.

4. The sling of claim 3 wherein the encircling strap is designed and adapted to fit no closer than about 1 centimeter from the neck of the wearer.

5. The sling of claim 3 wherein the encircling strap is designed and adapted to fit no closer than about 2 centimeters from the neck of the wearer.

6. The sling of claim 3 wherein the encircling strap is designed and adapted to fit no closer than about 3 centimeters from the neck of the wearer.

7. The sling of claim 3 wherein the encircling strap is designed and adapted to fit no closer than about 4 centimeters from the neck of the wearer.

8. The sling of claim 3 wherein the encircling strap is designed and adapted to fit no closer than about 5 centimeters from the neck of the wearer.

9. The sling of claim 3 wherein the encircling strap is designed and adapted to fit no closer than about 6 centimeters from the neck of the wearer.

10. The sling of claim 1 wherein the sling further comprises a shoulder pad designed and adapted to rest on the wearer's shoulder at a point of contact of the encircling portion and the clavicle of the wearer.

11. The sling of claim 1 wherein the encircling strap comprises a buckle, a hook portion and a loop portion or other means for adjusting the circumference of the encircling strap.

12. The sling of claim 1 wherein the first chest strap comprises a buckle, a hook portion and a loop portion or an other means for adjusting the supporting length of the first chest strap.

13. The sling of claim 1 wherein the back strap comprises a buckle, a hook portion and a loop portion or an other means for adjusting the supporting length of the back strap.

14. The sling of claim 1 wherein the sling further comprises a second chest strap having a first end connected to the front-facing side of the encircling strap, a second end connected to the anterior portion of the pouch and a middle portion supported by the first chest strap.

15. The sling of claim 14 wherein the first chest strap and the second chest strap connect at a point between the encircling strap and the anterior portion of the pouch.

16. The sling of claim 1 wherein the encircling strap consists essentially of a single strap.

17. The sling of claim 1 wherein the encircling strap consists essentially of a single strap comprising two dog leg bends.

18. The sling of claim 11 wherein the encircling strap further comprises a buckle, a hook portion and a loop portion or an other means for adjusting the circumference of the encircling portion.

19. The sling of claim 11 wherein the encircling strap further comprises a shoulder pad designed and adapted to rest on the wearer's shoulder at a point of contact of the encircling portion and the clavicle of the wearer.

20. A sling for supporting a portion of an arm of an individual, the sling comprising:
a means for receiving and supporting a portion of an arm of an individual, the means for receiving and supporting a portion of an arm of an individual having a posterior portion and an anterior portion;
a means for securing the sling to an individual, the means for securing the sling to an individual designed and adapted to encircle a shoulder, the means for securing the sling to an individual having a front-facing side and a back-facing side;
a first means for attaching the means for receiving and supporting a portion of an arm of an individual to the means for securing the sling to an individual; and
a second means for attaching the means for receiving and supporting a portion of an arm of an individual to the means for securing the sling to an individual.
